(19) United States
(12) Patent Application Publication
(10) Pub. No.: US 2026/0053489 A1
(43) Pub. Date: Feb. 26, 2026

(54) DEVICE FOR SUPPORTING AND POSITIONING A SURGICAL RETRACTOR BLADE ON A RING RETRACTOR

(71) Applicant: UNIVERSITA' DEGLI STUDI DELL'AQUILA, L'Aquila (IT)

(72) Inventor: Marco CLEMENTI, Roma (IT)

(21) Appl. No.: 19/104,181

(22) PCT Filed: Jul. 25, 2023
(86) PCT No.: PCT/IT2023/050175

§ 371 Date: Feb. 14, 2025

(30) Foreign Application Priority Data
Aug. 18, 2022 (IT) 102022000017409

(51) Int. Cl. A61B 17/02; A61B 17/00

(52) U.S. Cl. CPC A61B 17/0293; A61B 2017/00477

(57) ABSTRACT

A device to connect a surgical retractor blade to a superficial ring retractor.

### TECHNICAL FIELD

The present invention belongs to the technical field of surgical instruments that are used to retract and divaricate biological tissues in order to facilitate the vision of the surgical site and facilitate the access to the operating field.

In particular, the present invention describes a device for joining a surgical wound edge retractor with a retractor blade that retracts and retains internal viscera, thereby maintaining the advantages of a skin retractor (protection and homogeneous retraction of wound edges) but adding the advantages peculiar to a deep autostatic retractor.

### PRESENT STATUS OF THE ART

Surgeons use a wide range of instruments generically called “retractors”, normally used to separate the edges of an incision or surgical wound in order to achieve adequate tissue divarication and expose the surgical field, i.e., the area of the body that is to be subjected to surgery, allowing a broader view of the same.

It should be noted that in this patent text, the terms “surgical wound” and “incision” are used indifferently to refer to a gap in the skin and underlying biological tissues, usually made for therapeutic purposes.

Surgical retractors come in many shapes and sizes depending on their intended use and the type of tissue that needs to be retracted. They are instruments conformed in such a way as to avoid traumatization and can be either disposable or reusable, in the latter case they are made of materials suitable for sterilization, e.g., stainless steel.

These surgical instruments fall into two main categories: superficial skin and/or wall retractors, also known as superficial retractors, which are particularly suitable for keeping the edges of a wound or the edges of an incision separated, and deep retractors, the latter of which are designed to hold internal organs and forcibly force the separation of deeper biological tissues by overcoming the resistance of these tissues to divarication.

Both manual retractors, i.e., instruments that must be manually held by an operator, and autostatic retractors are known on the market; the latter are capable of providing stable retraction without requiring the operator to constantly maintain a manual traction.

In the field of superficial retractors, so-called double-ring wound retractors are known on the market. Such instruments allow atraumatic all-around divarication of the superficial tissues of the surgical access while simultaneously protecting the wound walls. They are devices conformed as a sleeve comprising two rings of soft plastic material joined circumferentially by a flexible tubular membrane of transparent plastic material. The cross section of each ring is generally circular, in other words, each ring is conformed like a torus, or doughnut. A first ring of the retractor is inserted inside the wound while the second ring remains on the outside of it; taking advantage of the elasticity and deformability of the material that makes up the outer ring, the operator rotates it on itself so that the tubular membrane gradually wraps around it until it reaches the edge of the wound itself, in this way the tubular membrane adheres to the side walls of the incision, keeping it spread apart and with the edges protected.

Superficial devices of this type are very practical and versatile, but they do not allow adequate divarication of deep tissues and internal organs and do not provide a complete view of the surgical site.

Deep retractors, on the contrary, are instruments that can ensure stable divarication of deep tissues and internal organs, however, especially in the case of autostatic retractors, they are very bulky, heavy and not practical to handle in addition to taking a long time to be placed. Finally, instruments of this type do not provide adequate protection of the surgical wound margins.

An example of an autostatic retractor, suitable for being connected to the rail of an operating table, is described in U.S. patent application U.S. Pat. No. 3,810,462A.

Among autostatic retractors, one of the most complete and widely used instruments is the Bookwalter® retractor. Such an instrument consists of a plurality of elements: a support system consists of a horizontal bar connected by means of a joint to a vertical bar attached to a dedicated rail of the operating table by means of a clamp; the horizontal bar is connected to a metal flat ring of circular, oval or malleable shape by means of a joint that allows an albeit limited adjustment of the inclination, so that its position can be adapted to the anatomical conformation of the patient and his position on the operating bed; one or more retractor blades are connected to the metal flat ring by means of a respective coupling that makes the stem of each retractor blade integral with the flat ring itself.

Such an autostatic retraction system is described in U.S. patent application U.S. Pat. No. 4,254,763A.

Instruments such as the one just described are very expensive as well as bulky, and their proper placement is time-consuming, which is incompatible with the urgency sometimes required in the case of surgeries to be performed in emergency situations.

Another disadvantage of these types of instruments is that in order to use them, it is necessary to have an operating table specially designed for connection with an instrument of the afore-mentioned type.

Patent application U.S. Pat. No. 20,141,71748A1 describes an assembly for positioning a retractor blade on a surgical support, which in the attached figures is depicted as a flat rim that fits into a slot of a rotating indexer. The assembly is attached to the rim of the surgical support only via a small spring-loaded retention clip, which, however, does not permit sufficiently firm locking and position of the assembly.

Patent application U.S. Pat. No. 20,031,99738A1 describes a clamp for connecting surgical instruments on a support member, the latter again is depicted in the figure as a flat rail connected to an operating table. The clamp is conformed in such a way that once it has been placed and clamped on the support rail, it is no longer possible to move the respective surgical instrument unless the entire clamp is disengaged.

Patent application WO2022044055A1 describes a surgical ring retractor device to which a plurality of supports for respective retractor elements are reversibly attached. Again, each support is conformed in such a way that once it has been positioned and tightened onto the ring of the retractor device, it is no longer possible to move the respective retractor element unless the support is also disengaged.

### OBJECTS AND SUMMARY OF THE INVENTION

It should be noted that in the present patent text the term “retractor blade” is generically understood to mean a surgical instrument of various shapes consisting of a flattened part, in the form of a spatula, suitable for divaricating tissues and a handle substantially orthogonal to the flattened part of the instrument. The handle is generally conformed as an elongated, slender stem, either smooth or indented; surgical retractor blades consisting of two pieces, i.e., a spatula and a supporting arm that can be reversibly coupled together, for example, by means of a male-female attachment, are also known on the market.

The objective of the present invention is to provide a device for using a superficial retractor that provides adequate retraction and protection of the wound walls, in association with a deep retractor blade, to allow a stable and optimal view of the surgical site.

These and other objectives are achieved by a device for connecting a surgical deep retractor blade to a superficial skin retractor, specifically to a ring wound retractor. The device is extremely quick to position, easy to handle, not bulky, inexpensive, and usable in any surgical setting in which a superficial retractor of the above-described type is positioned.

The innovative device which is the object of the present invention comprises a clamp suitable for accommodating the outer ring of a superficial skin retractor. Said clamp is suitable for supporting a surgical retractor blade whose stem is housed in a longitudinal cavity defined in said clamp.

Advantageously, the device subject of the present invention can support both retractor blades with a smooth stem and retractor blades with a notched stem and also support arms of re-tractor blades composed of two separate parts.

Very advantageously, the same reversible means that connect the clamp to the ring of the superficial retractor make it possible to position the device at any point on the ring of the superficial retractor and according to any inclination with respect to the plane on which the ring itself lies, so that it can be adapted to the conformation of the surgical site.

The surgical retractor blade is reversibly connected to said clamp by means of constraining means that either alternately lock the stem of the retractor blade preventing any movement or allow the retractor blade to be moved and rotated around its axis in order to position it optimally with respect to the operating field.

Preferably, such constraining means are of the self-locking type, particularly suitable when using retractor blades with indented stems, or include a quick release clamping system with a clamp, particularly suitable for smooth stem retractor blades.

In a preferred embodiment, such a clamp comprises an upper jaw and a lower jaw that tighten against the outer ring of the superficial retractor via a screw, spring or ratchet clamping system. Other reversible mechanisms of known types can be provided for quick connection.

The cavity defined longitudinally in the clamp has a cross-sectional area suitable for accommodating the stem of a surgical retractor blade, the latter extending inward from the ring of the retractor; in other words, when a retractor blade is connected to the clamp, the flattened part of the retractor blade is within the vertical projection of the ring of the retractor. Reversible means of connection of the stem with the clamp make it possible to modify the projection of the retractor blade from the rim of the ring of the retractor quickly and easily as needed.

A plurality of devices of the type described above, to each of which a respective retractor balde can be coupled, can be connected to the outer ring of a superficial skin retractor, each at a different position of the circumferential development of the ring and at any inclination with respect to the operating site.

### BRIEF DESCRIPTION OF THE DRAWINGS

FIG. 1 and FIG. 2 show two different perspective views of an embodiment of the device subject of the present invention especially suitable for supporting and positioning surgical retractor blades with indented stems. Also shown in the figures is the indented stem of a retractor blade, housed in the cavity (2) defined longitudinally in the clamp (1). The latter is formed by an upper half-body (11) and a lower half-body (12); the screw (13) passes vertically through the upper half-body (11) engaging the internal thread defined in the lower half-body (12). Also shown in FIG. 2 are the constraining means (3) for holding the stem of the retractor blade in place; in the embodiment shown said constraining means (3) comprise a self-locking pin with a horizontal axis housed in a corresponding cavity defined in the clamp (1).

FIG. 3 shows a front view of the device shown in FIGS. 1 and 2. The cross-sectional plane A-A is shown.

FIG. 4 shows the cross-sectional view according to the A-A plane shown in FIG. 3. The circumferential ridges and grooves of the indentation defined on the lateral surface of the stem of the retractor blade are visible.

FIG. 5 and FIG. 6 show two perspective views of a different embodiment of the device that is the subject of the present invention, in which the constraining means (3) include a quick-release clamp, suitable for supporting and locking smooth stem retractor blades in the most convenient position. Also shown in the figures is the smooth stem of a retractor blade, housed in the cavity (2) defined longitudinally in the clamp (1).

FIG. 7 shows a perspective view of the embodiment of the device shown in FIG. 1, mounted on the upper ring of a ring wound retractor. The upper half-body (11) and lower half-body (12) surround the upper ring of the wound retractor, tightening it, thanks to the action of the screw (13). An indented stem is housed in the cavity (2), which, at its distal end, supports the spatula of a retractor blade; the latter is shown dashed as it can have different shapes and sizes according to needs.

FIG. 8 shows a further perspective view of the device shown in FIG. 7; visible are the constraining means (3) designed to prevent movement of the stem of the retractor blade within the cavity (2), said constraining means are shown here in the form of a self-locking pin with a horizontal axis.

FIG. 9 shows a perspective view of the device according to the embodiment shown in FIG. 5, mounted on the upper ring of a ring wound retractor. Constraining means (3) comprising a quick-release clamp, suitable for supporting and locking the smooth-stem retractor blades in the most suitable position, are visible.

### DETAILED DESCRIPTION OF AN EMBODIMENT OF THE INVENTION

According to a preferred embodiment, the device for supporting and positioning surgical retractor blades on a ring wound retractor, which is the subject of the present invention, comprises a clamp (1) in which is defined a longitudinally extending cavity (2) in which the stem of a surgical retractor blade is slidingly engaged, said clamp (1) being conformed to be able to connect integrally with the upper ring of said superficial retractor.

In the embodiment described here, said longitudinal cavity (2) is conformed as a cylindrical cavity horizontally traversing the clamp (1) and is suitable to accommodate the stem of a common type of surgical retractor blade, either indented or smooth. Other constructional forms of the cavity (2) are, of course, possible; the latter can be shaped to accommodate retractor blades with smooth or indented stems of various sizes and shapes, e.g., stems with a cylindrical cross-section or with a rectangular cross-section or other shape. The stem of the surgical retractor blade is inserted into the cavity (2) by sliding it until the desired protrusion of the spatula of the retractor blade from the ring of the retractor is reached, after which the retractor blade is locked in the desired position by appropriate reversible constraining means (3).

These constraining means (3) are movable between an engaged position in which they block translation and/or rotation of the stem of the surgical retractor blade relative to the clamp (1) and a rest position, in which the retractor blade is free to translate relative to the clamp (1) and/or to rotate relative to its longitudinal axis, so that the position of the retractor blade relative to the ring of the retractor can be changed.

In the attached figures some possible embodiments of movable constraining means (3) are shown; other types of movable constraining means capable of blocking the translation and/or rotation of the stem of the retractor blade are, of course, possible.

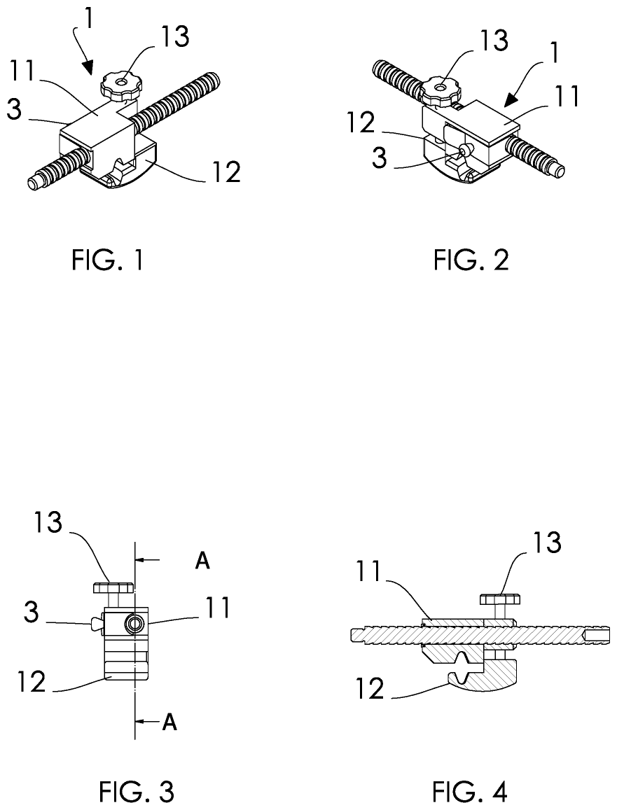

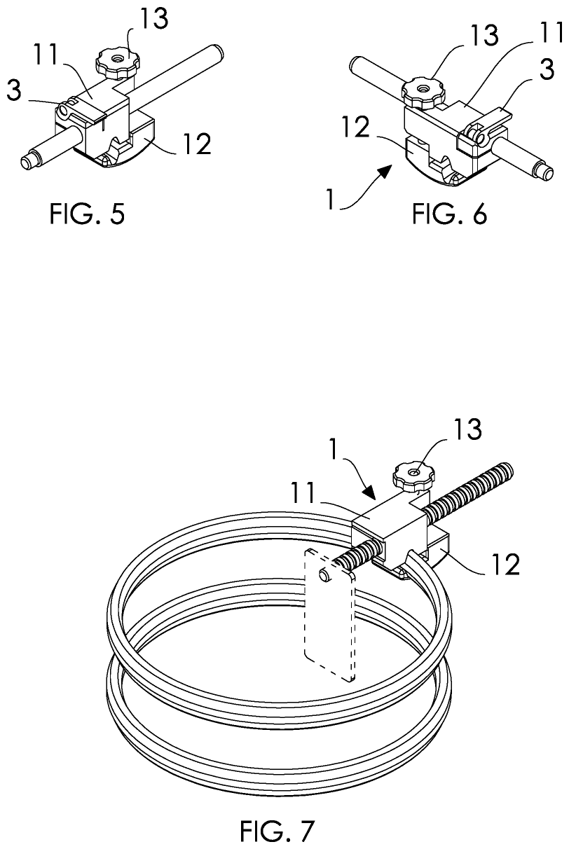

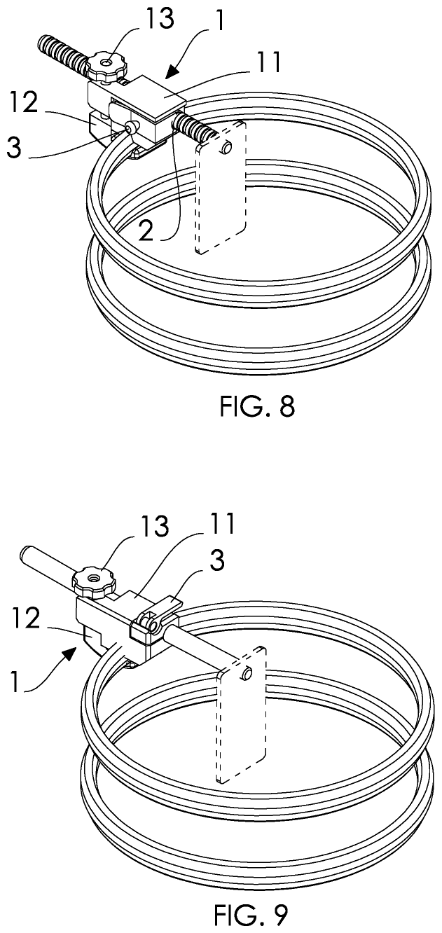

In the embodiment shown in FIGS. 1 and 2, said constraining means (3) are represented by a self-locking pin with a horizontal axis housed in a corresponding cavity defined in the clamp (1). When in the engaged position, said pin goes against the lateral surface of the stem of the surgical retractor blade, blocking its movement relative to the clamp (1). If retractor blades with indented stems are used, when the constraining means (3) are in the engaged position said self-locking pin fits into the circumferential groove defined between two consecutive circumferential ridges of the indentation defined on the lateral surface of the stem of the retractor blade itself.

Alternatively, such constraining means (3) may include a quick-release clamp operated by an eccentric cam clamping lever; in the engaged position said quick-release clamp tightens around the lateral surface of the stem of the retractor blade. This embodiment, shown in FIG. 3, is particularly advantageous when using smooth stem retractor blades.

In the embodiment described here, the clamp (1) comprises an upper half-body (11) and a lower half-body (12) that are mutually cooperating and designed to encircle the ring of the retractor on the upper and lower sides, restraining and clamping it. The lower portion of the upper half-body (11) is conveniently conformed to adhere to the upper surface of the ring of the retractor while the upper portion of the lower half-body (12) is conformed to adhere to the lower surface of the ring of the retractor; reciprocal clamping means (13) allow the upper half-body (11) to be brought closer to the lower half-body (12) and vice versa. In this configuration, the upper half-body (11) and the lower half-body (12) form a prismatic pair, so that one of the two half-bodies (11, 12) can be moved with translatory motion relative to the other, by means of said reciprocal clamping means (13). In practice, when the device is mounted on a retraction ring, the upper half-body (11) and the lower half-body (12) encircle the outer ring of the retractor superiorly and inferiorly, clamping it as in a vise.

In the embodiment shown in the figures, the lower portion of the upper-half body (11) and the upper portion of the lower-half body (12) are each conformed as a portion of the side surface of a cylinder, so as to fit with the conformation of the ring of the retractor, having a circular cross-sectional area.

In this embodiment, the longitudinal cavity (2) suitable for accommodating the stem of the surgical retractor blade is defined in the upper half-body (11) of the clamp (1). Of course, other embodiments are possible in which the cavity (2) is defined in the lower half-body (2) of said clamp (1).

The reciprocal clamping means (13) of the two half-bodies (11, 12) include a vertical-axis screw that passes through the upper half-body (11), forming a revolute joint with it, and engages a corresponding thread defined in the lower half-body (12), forming a helical joint with it. Other clamping means such as a cam or similar means may be used in place of the screw.

1. A device for supporting and positioning a surgical retractor blade on a surgical ring retractor, said retractor blade comprising a stem, said device comprising a clamp (1) adapted to connect reversibly with the outer ring of a surgical ring retractor and comprising means for reversibly supporting the stem of a surgical retractor blade protruding inwardly from the ring of the retractor characterized in that said clamp (1) comprises a mutually cooperating upper half-body (11) and lower half-body (12) respectively adapted to surround superiorly and inferiorly the surgical retractor ring, and in that said clamp (1) is traversed longitudinally by a cavity (2) adapted to accommodate the stem of said surgical retractor blade, said cavity (2) being defined in the upper half-body (11) of said clamp (1), the latter includes constraining means (3) adapted to reversibly block the movement of the stem of said retractor blade.

2. Device according to claim 1 characterized in that said constraining means (3) are movable between a resting position, in which said retractor blade is free to translate with respect to said clamp (1) and to rotate with respect to its longitudinal axis, and an engaged position, in which said constraining means (3) prevent the movement of said retractor blade with respect to said clamp (1).

3. Device according to claim 1 characterized in that said constraining means (3) comprise a self-locking pin with a horizontal axis housed in a corresponding cavity defined in the clamp (1), said pin being capable of contrasting the lateral surface of the stem of said surgical retractor blade.

4. Device according to claim 1 characterized in that said constraining means (3) comprise a quick release clamp comprising an eccentric cam lever, in the engaged position said quick release clamp is tightened around the lateral surface of the stem of said surgical re-tractor blade.

5. Device according to claim 1 characterized in that said clamp (1) includes reciprocal clamping means (13) of said upper half-body (11) and lower half-body (12) the latter forming a prismatic pair, so that by acting on said reciprocal clamping means (13), one of said half-body (11, 12) can be moved with translatory motion with respect to the other.

6. Device according to claim 5 characterized in that said reciprocal clamping means (13) comprise a vertical-axis screw that passes through the upper half-body (11) forming a turning pair with it, engaging a corresponding female thread defined in the lower half-body (12).

7. Device according to claim 1 characterized in that the lower portion of the upper half-body (11) and the upper portion of the lower half-body (12) are each conformed as a portion of the lateral surface of a cylinder.

8. Device according to claim 1 characterized in that it is made of material suitable for being sterilized.
